(19) United States
(12) Patent Application Publication
(10) Pub. No.: US 2020/0179568 A1
(43) Pub. Date: Jun. 11, 2020

(54) METHOD FOR PRODUCING DECELLULARIZED TISSUE, DECELLULARIZED TISSUE, AND APPARATUS FOR PRODUCING DECELLULARIZED TISSUE

(71) Applicants: Satoshi SHINOHARA, Shizuoka (JP); Shogo SUZUKI, Kanagawa (JP); Shogo TORII, Shizuoka (JP)

(72) Inventors: Satoshi SHINOHARA, Shizuoka (JP); Shogo SUZUKI, Kanagawa (JP); Shogo TORII, Shizuoka (JP)

(21) Appl. No.: 16/611,035

(22) PCT Filed: Mar. 29, 2018
(86) PCT No.: PCT/JP2018/013272

§ 371 Date: Nov. 5, 2019

(30) Foreign Application Priority Data
May 11, 2017 (JP) 2017-095004

(51) Int. Cl. A61L 27/36

(52) U.S. Cl. CPC A61L 27/3687; A61L 27/3608; A61L 27/3691; A61L 27/3625; A61L 27/3629; A61L 27/3612

(57) ABSTRACT

A method for producing a decellularized tissue includes steps of lysing a cell of a biological tissue using a liquid containing a liquefied gas, and degrading a nucleic acid component contained in the lysed cell of the biological tissue using a nucleolytic enzyme.

### TECHNICAL FIELD

The present invention relates to a method for producing a decellularized tissue, a decellularized tissue, and an apparatus for producing a decellularized tissue.

### BACKGROUND ART

In the field of regenerative medicine, a decellularized tissue, which is produced by removing cellular components, such as cytoplasmic components, cytosolic components, cytoskeleton, and cell membrane components, from a biological tissue of a human or some other mammal, is used for implantation as a scaffold tissue for regenerating a failed organ of a patient, for example. A decellularized tissue is mainly composed of extracellular matrix components, such as elastin, collagen (type I, type IV), and laminin.

Conventional methods for producing a decellularized tissue involve decellularizing a biological tissue using a treatment liquid containing a surfactant (see, e.g., Patent Literature Documents 1-3). Specifically, the biological tissue is immersed in a treatment liquid containing a surfactant for several days while being subjected to agitation. However, because surfactants cause degradation of proteins constituting extracellular matrix components, the decellularized tissue may be damaged. Also, decellularization using the above method requires time and residual surfactant may remain in the decellularized tissue.

In this respect, a method of decellularizing a biological tissue using supercritical carbon dioxide is known (see, e.g., Patent Literature Document 4).

However, in this method, a large amount of DNA remains in the decellularized tissue.

Note that the amount of DNA per dry weight of the decellularized tissue is desirably less than 50 ng/mg (see, e.g., Non-Patent Literature Document 1).

### CITATION LIST

### Patent Literature

[PTL 1] Japanese Translation of PCT International Application Publication No. JPT-2005-514971
[PTL 2] Japanese Translation of PCT International Application Publication No. JPT-2006-507851
[PTL 3] Japanese Translation of PCT International Application Publication No. JPT-2005-531355
[PTL 4] Japanese Unexamined Patent Application Publication No. 2007-105081

### Non Patent Literature

[NPL 1] Biomaterials. 2011 April; 32 (12): 3233-3243

### SUMMARY OF INVENTION

### Technical Problem

One object of the present invention is to provide a method for producing a decelluralized tissue that is substantially free of damage and has an amount of DNA per dry weight of less than 50 ng/mg.

### Solution to Problem

According to one aspect of the present invention, a method for producing a decelluralized tissue includes steps of lysing a cell of a biological tissue using a liquid containing a liquefied gas, and degrading a nucleic acid component contained in the lysed cell of the biological tissue using a nucleolytic enzyme.

### Advantageous Effects of Invention

According to one aspect of the present invention, a method for producing a decelluralized tissue that is substantially free of damage and has an amount of DNA per dry weight of less than 50 ng/mg can be provided.

### BRIEF DESCRIPTION OF DRAWINGS

FIG. 1 is a flowchart illustrating an example method for producing a decelluralized tissue according to an embodiment of the present invention.

FIG. 2 is a graph representing a saturated vapor pressure curve of dimethyl ether.

FIG. 3 is a schematic diagram illustrating an example decellularization pretreatment apparatus according to an embodiment of the present invention.

FIG. 4 is a schematic diagram illustrating another example decellularization pretreatment apparatus according to an embodiment of the present invention.

FIG. 5 is an optical micrograph of a decelluralized tissue of Example 1 stained with hematoxylin and eosin.

FIG. 6 is an optical micrograph of a decellularized tissue of Example 2 stained with hematoxylin and eosin.

FIG. 7 is an optical micrograph of a decellularized tissue of Example 3 stained with hematoxylin and eosin.

FIG. 8 is an optical micrograph of an untreated biological tissue stained with hematoxylin and eosin.

### DESCRIPTION OF EMBODIMENTS

In the following, embodiments of the present invention will be described with reference to the accompanying drawings.

(Method for Producing Decelluralized Tissue)

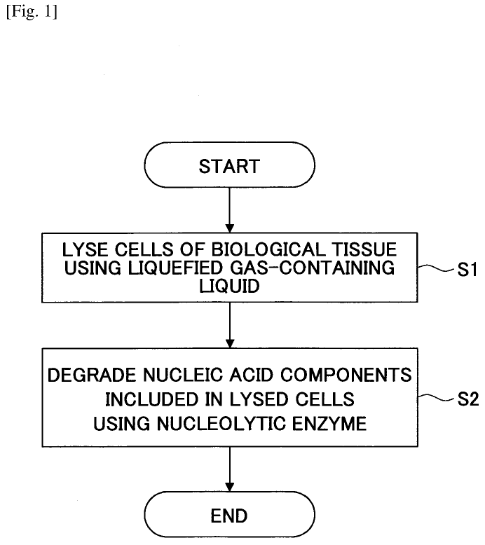

FIG. 1 illustrates an example method for producing a decelluralized tissue according to an embodiment of the present invention.

The method for producing a decelluralized tissue includes a step of lysing (breaking down) cells of a biological tissue using a liquid containing a liquefied gas (step S1), and a step of degrading nucleic acid components contained in the lysed cells using a nucleolytic enzyme (nuclease) (step S2).

In step S1, for example, a biological tissue may be brought into contact with a liquid containing a liquefied gas to lyse (break down) the cells of the biological tissue and expose nucleic acid components to the cell exterior. In this way, the decellularized tissue may be substantially free of damage, and the liquefied gas may be less likely to remain in the decellularized tissue. In the present embodiment, the liquefied gas-containing liquid dissolves cell membrane components and can therefore lyse (break down) the cells.

In the present description, liquefied gas refers to the liquid form of a substance that constitutes a gas at normal temperature and normal pressure (0° C., 1 atm (0.101325 MPa)).

Although the liquefied gas used in the present embodiment is not particularly limited as long as it is capable of lysing the cells of a biological tissue, for example, dimethyl ether, ethyl methyl ether, formaldehyde, ketene, acetaldehyde, propane, butane, liquefied petroleum gas, or a combination of two or more of the above substances may be used. Among the above substances, ethyl methyl ether and dimethyl ether may be suitably used in view of the fact that they can be liquefied at a relatively low temperature and low pressure. In particular, dimethyl ether may be preferably used.

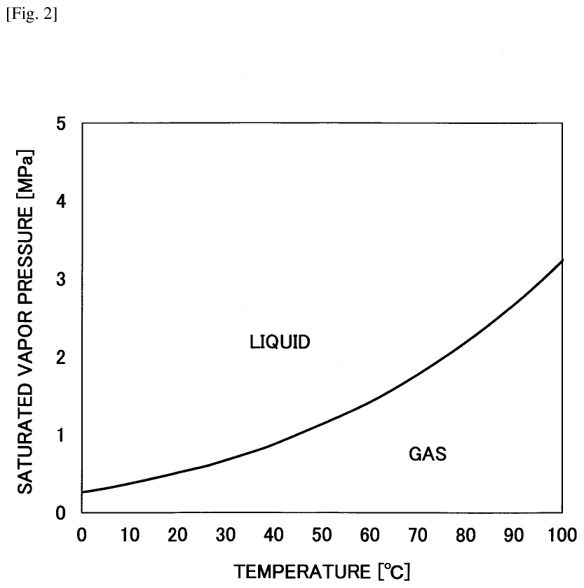

Dimethyl ether is liquefied at about 1° C. to 40° C. and about 0.2 MPa to 5 MPa (see FIG. 2), and as such, a relatively low-cost apparatus for producing a decellularized tissue manufacturing apparatus may be used. Also, because liquefied dimethyl ether easily evaporates under normal temperature and normal pressure, it is less likely to remain in the decellularized tissue.

The process of step S1 may be carried out in an environment above a saturated vapor pressure in an airtight extraction tank, for example, in order to maintain the liquid state of the liquefied gas.

Although the method used for bringing the biological tissue into contact with the liquefied gas-containing liquid is not particularly limited, for example, a suitable method may be selected based on the nature and property of the biological tissue, such as a method of mixing and stirring the liquefied gas-containing liquid and the biological tissue, a method of immersing the biological tissue in the liquefied gas-containing liquid, or a method of circulating the liquefied gas-containing liquid and bringing it into contact with the biological tissue.

The liquefied gas-containing liquid may further contain a solvent (entrainer).

Although the solvent used is not particularly limited, for example, ethanol, water, a physiological saline solution, PBS (phosphate buffered physiological saline), or a combination of two or more of the above substances may be used.

The amount of solvent added is preferably adjusted to be less than or equal to its solubility in the liquefied gas. In this way, uniformity of the liquefied gas-containing liquid may be achieved.

The temperature of the liquefied gas is preferably in the range from 1° C. to 40° C., and more preferably in the range from 10° C. to 30° C. When the temperature of the liquefied gas is in the range from 1° C. to 40° C., the cost of a decellularization pretreatment apparatus as described below can be reduced.

The pressure of the liquefied gas is preferably in the range from 0.2 MPa to 5 MPa, and more preferably in the range from 0.3 MPa to 0.7 MPa. When the pressure of the liquefied gas is in the range from 0.2 MPa to 5 MPa, the cost of the decellularization pretreatment apparatus as described below can be reduced.

Although the biological tissue is not particularly limited, for example, the biological tissue may be soft tissue, such as the skin, blood vessel, valvular heart valve, cornea, amnion, dura, or a part thereof, obtained from a human or some other mammal; an organ, such as the heart, kidney, liver, pancreas, brain, or a part thereof, obtained from a human or some other mammal; or connective tissue, such as the bone, cartilage, tendon, or a part thereof, obtained from a human or some other mammal.

After bringing the biological tissue into contact with the liquefied gas-containing liquid and then adjusting the temperature and pressure back to normal temperature and normal pressure, the liquefied gas is vaporized and removed.

Note that when cell lysis is not adequately performed by carrying out the process of step S1 only once, the process of step S1 may be repeated multiple times.

In step S2, for example, the biological tissue that has been subjected to the cell lysis process of step S1 may be brought into contact with a solution containing a nucleolytic enzyme to cause degradation of nucleic acid components exposed to the cell exterior. As a result, the amount of DNA per dry weight of the decellularized tissue may be less than 50 ng/mg.

Although the nucleolytic enzyme used in the present embodiment is not particularly limited as long as it is capable of degrading DNA, DNase (e.g., DNase I) may be used, for example.

Although the method used for bringing the lysed cells into contact with the nucleolytic enzyme-containing solution is not particularly limited, example methods that may be used include a method of mixing and stirring the nucleolytic enzyme-containing solution and the biological tissue that has been subjected to the cell lysis process, a method of immersing the biological tissue that has been subjected to the cell lysis process in the nucleolytic enzyme-containing solution, and a method of circulating the nucleolytic enzyme-containing solution and bringing it into contact with the biological tissue that has been subjected to the cell lysis process.

The method used for bringing the lysed cells into contact with the nucleolytic enzyme-containing solution may be appropriately selected based on the nature and property of the biological tissue that has been subjected to the cell lysis process.

Note that in some embodiments, the process of step S2 may be included in the process of step S1. That is, cells of a biological tissue may be lysed and the nucleic acid components included in the cells may be degraded using a liquefied gas-containing liquid and a nucleolytic enzyme, for example. In this case, a nucleolytic enzyme-containing solution may be introduced while the biological tissue is in contact with the liquefied gas-containing liquid, for example.

In a preferred embodiment, the method for producing a decellularized tissue further includes a step of washing the biological tissue that has been subjected to the nucleic acid component degradation process (step S3).

In step S3, for example, the biological tissue that has been subjected to the nucleic acid component degradation process of step S2 may be brought into contact with a washing solution to be washed.

Examples of the washing solution include water, a physiologically compatible liquid, an aqueous solution of a physiologically acceptable organic solvent, a liquefied gas-containing liquid, and the like.

Although the physiologically compatible liquid is not particularly limited, examples include a physiological saline solution, PBS (phosphate buffered physiological saline), and a combination of two or more the above substances. In particular, a physiological saline solution may be preferably used, for example.

Although the physiologically acceptable organic solvent is not particularly limited, ethanol and the like may be used, for example.

The liquefied gas-containing liquid may be the same as or different from the liquefied gas-containing liquid used in step S1.

Note that in some embodiments, the process of step S3 may involve washing the biological tissue that has been subjected to the nucleic acid component degradation process with an aqueous solution of a physiologically acceptable organic solvent or a liquefied gas-containing liquid before washing the biological tissue with water or a physiologically compatible liquid, for example.

Although the method used for bringing the biological tissue that has been subjected to the nucleic acid component degradation process into contact with the washing solution is not particularly limited, example methods that may be used include a method of mixing and stirring the washing solution and the biological tissue that has been subjected to the nucleic acid component degradation process, a method of immersing the biological tissue that has been subjected to the nucleic acid component degradation process in the washing solution, and a method of circulating the washing solution and bringing it into contact with the biological tissue that has been subjected to the nucleic acid component degradation process.

The method of bringing the biological tissue that has been subjected to the nucleic acid component degradation process into contact with the washing solution may be appropriately selected based on the nature and property of the biological tissue that has been subjected to the nucleic acid component degradation process.

Note that the biological tissue that has been subjected to the nucleic acid component degradation process is preferably washed with the washing solution at a temperature of 4° C. to 40° C.

In the case of washing the biological tissue that has been subjected to the nucleic acid component degradation process with a liquefied gas-containing liquid, the washing process is preferably carried out in an environment under at least a saturated vapor pressure, such as in an airtight extraction tank, for example, in order to maintain the liquid state of the liquefied gas.

Note that the process time of the process of washing the biological tissue that has been subjected to the nucleic acid component degradation process with the washing solution is not particularly limited as long as washing is performed over a sufficient period of time for adequately removing the enzyme used in step S2 and the cellular components exposed to the cell exterior in step S1.

Note that in some embodiments, when washing the biological tissue that has been subjected to the nucleic acid component degradation process with the washing solution, the washing solution may be exchanged and the washing process may be repeatedly performed, for example.

By implementing the method for producing a decellularized tissue according to the present embodiment, a decellularized tissue that is substantially free of damage and has an amount of DNA per dry weight of less than 50 ng/mg can be obtained. When the amount of DNA per dry weight of the decellularized tissue is less than 50 ng/mg, immune reactions may be prevented when the decelluralized tissue is transplanted into a living body.

(Decellularization Pretreatment Apparatus)

The decellularization pretreatment apparatus used in the present embodiment is not particularly limited as long as it is capable of lysing cells of a biological tissue using a liquefied gas-containing liquid.

In the following, an example case where liquefied dimethyl ether is used as the liquefied gas-containing liquid will be described.

The decellularization pretreatment apparatus may lyse cells of a biological tissue by bringing the biological tissue into contact with liquefied dimethyl ether that is produced by pressurizing dimethyl ether to at least its saturated vapor pressure in an extraction tank, for example. Also, the decellularization pretreatment apparatus may vaporize the liquefied dimethyl ether by reducing the pressure to less than the saturated vapor pressure to remove the liquefied dimethyl ether from the biological tissue that has been subjected to the cell lysis process.

Specifically, the decellularization pretreatment apparatus includes a liquid delivery unit (a) for delivering liquefied dimethyl ether from a storage unit (g) to a contact unit (b), the contact unit (b) for bringing a biological tissue into contact with liquefied dimethyl ether, and a derivation unit (c) for deriving the liquefied dimethyl ether that has come into contact with the biological tissue from the contact unit (b). Also, the decellularization pretreatment apparatus includes a separation unit (d), which may be a separation tank for separating dimethyl ether by adjusting the temperature and pressure or a membrane separation tank for separating dimethyl ether through membrane separation, and a condensing unit (e) for condensing dimethyl ether by adjusting the temperature and pressure. Further, the decellularization pretreatment apparatus includes a vaporization unit (f) for vaporizing the liquefied dimethyl ether by adjusting the temperature and pressure, the storage unit (g) for storing the liquefied dimethyl ether, a supply unit (h) for supplying liquefied dimethyl ether, and a detection unit (i) for detecting the temperature and pressure.

Although the liquid delivery unit (a) is not limited to a particular configuration as long as it is capable of adjusting the flow rate of the liquefied dimethyl ether, for example, a liquid delivery pump or a thermal drive may be used.

In the following, a decellularization pretreatment apparatus suitable for carrying out the process of step S1 will be described.

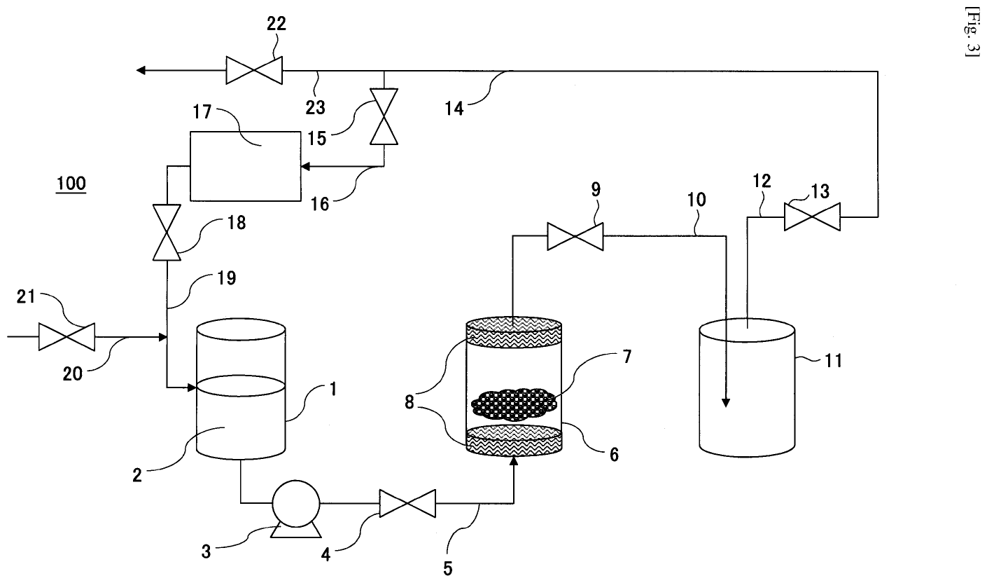

FIG. 3 illustrates an example configuration of a decellularization pretreatment apparatus 100 according to an embodiment of the present invention.

Note that FIG. 3 is merely a schematic illustration that facilitates understanding of the overall shape, size, and arrangement of component elements of the decellularization pretreatment apparatus according to the present embodiment. The present invention is in no way not limited by the following description, and component elements of the decellularization pretreatment apparatus can be suitably modified or changed within the scope of the present invention.

The decellularization pretreatment apparatus 100 includes a storage tank 1 for storing liquefied dimethyl ether 2, an extraction tank 6 for bringing a biological tissue 7 into contact with the liquefied dimethyl ether 2, a separation tank 11 for separating liquid derived from the extraction tank 6, and a pump 3 for delivering the liquefied dimethyl ether 2 from the storage tank 1 to the extraction tank 6. Also, the decellularization pretreatment apparatus 100 includes conduits 5, 10, 12, 14, 16, 19, and 20 for deriving or introducing (liquefied) dimethyl ether, and valves 4, 9, 13, 15, 18, and 21 for adjusting the air pressure in each tank and controlling the derivation and introduction of (liquefied) dimethyl ether. The pressure within the extraction tank 6 and the separation tank 11 can be adjusted in order to maintain the liquid state of the liquefied dimethyl ether.

In the decellularization pretreatment apparatus 100, the pump 3, the valve 4, and the conduit 5 for introducing the liquefied dimethyl ether 2 from the storage tank 1 to the extraction tank 6 serve as the liquid delivery unit (a). The extraction tank 6 serves as the contact unit (b). The conduit 10 and the valve 9 for deriving the liquefied dimethyl ether 2 from the extraction tank 6 serve as the derivation unit (c). Further, the separation tank 11 serves as the separation unit (d). The condenser 17 serves as the condensing unit (e). The conduit 12 and the valve 13 connected to the separation tank 11 serve as the vaporization unit (f). The storage tank 1 serves as the storage unit (g). The conduits 19 and 20 serve as the supply unit (h).

The decellularization pretreatment apparatus 100 may include further component elements, such as a thermometer and a pressure gauge for detecting the temperature and pressure in each tank, a stirrer for stirring the contents of each tank, and a device for circulating an inert gas (e.g., nitrogen) for purging an active gas (e.g., oxygen) within the tanks and conduits, for example.

In the following, a method of carrying out the process of step S1 using the decellularization pretreatment apparatus 100 will be described.

First, the biological tissue 7 is introduced into the extraction tank 6 that has filters 8 arranged at its upstream side and downstream side. At this time, the valves 4, 9, 13, 15, 18, 21, 22 are closed. Note that when an adequate amount of liquefied dimethyl ether 2 is not stored in the storage tank 1, the valve 21 is opened and liquefied dimethyl ether 2 is supplied to the storage tank 1 via the conduit 20 after which the valve 21 is closed. At this time, the valve 18 may be opened and closed along with the valve 21. Note that liquefied dimethyl ether is produced by pressurizing dimethyl ether to at least its saturated vapor pressure (see FIG. 2).

Then, the valve 4 is opened, and the liquefied dimethyl ether 2 in the storage tank 1 is withdrawn by the pump 3, introduced via the conduit 5 into the extraction tank 6, and brought into contact with the biological tissue 7, after which the valve 4 is closed. As a result, phospholipids that are the main components of the cell membranes of the biological tissue 7 are dissolved, and the cells of the biological tissue 7 are lysed.

Then, the valves 4 and 9 are opened, the liquefied dimethyl ether is withdrawn from the extraction tank 6 by the pump 3 and introduced into the separation tank 11 via the conduit 10. As a result, the liquefied dimethyl ether in the extraction tank 6 having the phospholipids dissolved therein is introduced into the separation tank 11 via the conduit 10. At this time, because the filter 8 is arranged at the upstream side and downstream side of the extraction tank 6, the biological tissue 7 that has been subjected to cell lysis remains in the extraction tank 6.

The timing at which the valves 4 and 9 are opened is controlled such that a predetermined time period elapses from the time the liquefied dimethyl ether is introduced into the extraction tank 6 to enable the liquefied dimethyl ether to come into contact with the biological tissue 7. Note that while the liquefied dimethyl ether is in contact with the biological tissue 7, the liquefied dimethyl ether and the biological tissue 7 may be left at a standstill for a predetermined period of time or may be stirred, for example.

Then, the valve 4 is closed, the valves 9, 13 and 22 are opened, and the pressure is reduced to less than the saturated vapor pressure of dimethyl ether so that the liquefied dimethyl ether existing between the valve 4 and the valve 13 is vaporized and discharged from the conduit 23 via the conduit 14. Note that a pump may be used as necessary to discharge the dimethyl ether, for example. As a result, the biological tissue 7 that has been subjected to cell lysis remains in the extraction tank 6, and the phospholipids remain in the separation tank 11.

Note that when the valve 22 is closed and the valve 15 is opened, the vaporized dimethyl ether is introduced into the condenser 17 via the conduit 16. As a result, liquefied dimethyl ether produced by condensation of dimethyl ether can be recycled.

Although an example case where the liquefied dimethyl ether 2 in the storage tank 1 is discontinuously withdrawn has been described above, in other examples, the liquefied dimethyl ether 2 in the storage tank 1 may be continuously withdrawn.

Specifically, the valves 4 and 9 may be opened so that the liquefied dimethyl ether 2 in the storage tank 1 may be continuously introduced from the conduit 5 into the extraction tank 6, and the liquefied dimethyl ether in the extraction tank 6 having the phospholipids dissolved therein may be continuously withdrawn from the extraction tank 6 to the conduit 10. In this case, the internal structure of the extraction tank 6 is preferably configured so that the liquefied dimethyl ether comes into contact with the biological tissue 7.

Also, note that in some embodiments, the decellularization pretreatment apparatus may be configured to adjust the temperature instead of the pressure to liquefy the dimethyl ether (or vaporize the liquefied dimethyl ether).

(Nucleic Acid Component Degradation Apparatus)

A nucleic acid component degradation apparatus used in the present embodiment is not particularly limited as long as it is capable of degrading nucleic acid components contained in the cells lysed by the decellularization pretreatment apparatus using a nucleolytic enzyme. For example, a known agitator may be used.

Note that in some embodiments, the nucleic acid component degradation apparatus may be included in the decellularization pretreatment apparatus. In this case, for example, while the biological tissue 7 is in contact with the liquefied dimethyl ether in the extraction tank 6, a solution containing a nucleolytic enzyme may be introduced into the extraction tank 6 using a known method, for example.

(Washing Apparatus)

A washing apparatus used in the present embodiment is not particularly limited as long as it is capable of washing the biological tissue that has been subjected to nucleic acid component degradation process by the nucleic acid component degradation apparatus. For example, a known agitator may be used.

Note that in a case where the biological tissue that has been subjected to nucleic acid component degradation is washed with liquefied dimethyl ether, the decellularization pretreatment apparatus 100 can be used, for example.

### EXAMPLES

In the following, the present invention will be described more specifically with reference to specific working examples. However, the present invention is not limited to these examples.

### Example 1

(Step S1)

Using a decellularization pretreatment apparatus as illustrated in FIG. 4, cells of a porcine aorta 57 as an example of a biological tissue were lysed.

Specifically, the porcine aorta 57 that was cut into a slice of about 3 cm in thickness was placed in an extraction tank 56 that has filters 55 arranged at its upstream side and downstream side. Then, 60 mL of dimethyl ether 52 was filled into a storage tank 51, pressurized to 0.8 MPa, and liquefied. At this time, the temperature of a constant temperature tank 50 was set to 37° C. Dimethyl ether was placed in a separation tank 61 in advance, and valves 53, 54, 58, 59, and 60 were closed. Then, the valves 53, 54, 58, 59 were opened to cause the flow of liquefied dimethyl ether. When the extraction tank 56 was filled with the liquefied dimethyl ether, the valves 54, 58 were closed, and the porcine aorta 57 was immersed in the liquefied dimethyl ether. Then, the valves 54 and 58 were opened, the flow rate of liquefied dimethyl ether was adjusted to 10 mL/min by the valve 59, and the liquefied dimethyl ether having phospholipids dissolved therein was collected in the separation tank 61. Thereafter, the valve 59 was closed, the separation tank 61 was removed from the apparatus, and the liquefied dimethyl ether was volatilized under atmospheric pressure in a fume hood.

By repeating the above operation 10 times, the porcine arota 57 was brought into contact with 600 mL of liquefied dimethyl ether. Thereafter, the valve 54 was closed, the valves 58, 59, and 60 were opened, the pressure in the extraction tank 56 was brought to atmospheric pressure, and the liquefied dimethyl ether in the extraction tank 56 was volatilized and discharged. Then, the biological tissue that has been subjected to cell lysis was removed.

(Step S2)

The biological tissue that has been subjected to the cell lysis process of step S1 was placed in a physiological saline solution containing 0.2 mg/mL of DNase I (manufactured by Roche Diagnostics) and 0.05 M of MgCl₂ (manufactured by Wako Pure Chemical Industries, Ltd.) and was subjected to agitation for 7 days in an atmosphere at 4° C. to degrade DNA.

(Step S3)

The biological tissue that has been subjected to the DNA degradation process of step S2 was placed in a physiological saline solution containing 80% by volume of ethanol and subjected to agitation for 3 days in an atmosphere at 4° C. Then, the biological tissue was placed in a physiological saline solution and subjected to agitation for 1 day in an atmosphere at 4° C. to obtain a decellularized tissue.

The obtained decellularized tissue was stored in a physiological saline solution at 4° C.

### Examples 2 and 3

In Examples 2 and 3, decellularized tissues were obtained in the same manner as Example 1 except that in step S2, agitation of the biological tissues that were subjected cell lysis was conducted for 5 days and 3 days, respectively, in an atmosphere at 4° C.

The obtained decellularized tissues were stored in a physiological saline solution at 4° C.

### Example 4

In Example 4, a decellularized tissue was obtained in the same manner as Example 1 except that in step S1, 57 mL of dimethyl ether and 3 mL of water were used instead of 60 mL of dimethyl ether.

The obtained decellularized tissue was stored in a physiological saline solution at 4° C.

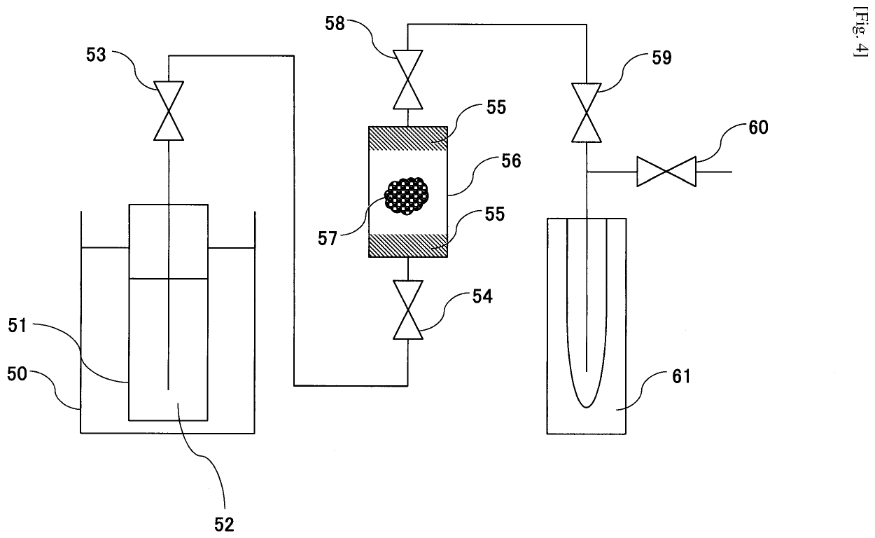

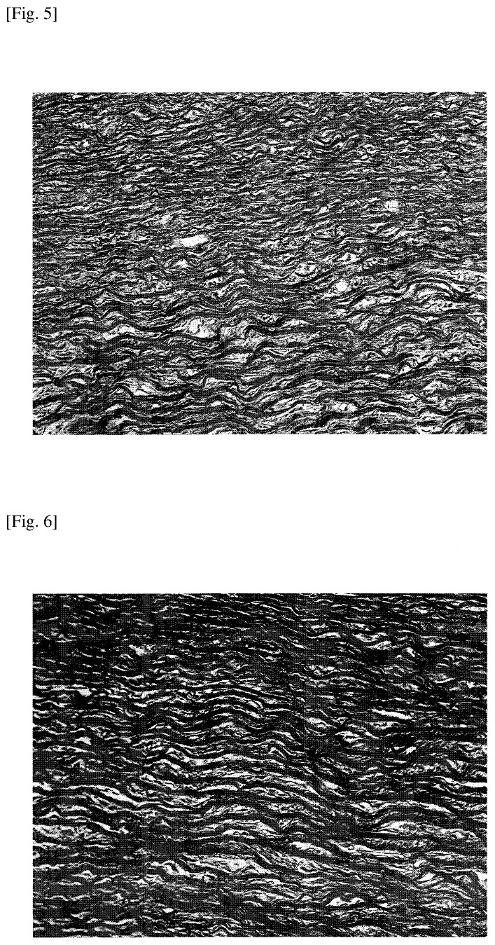

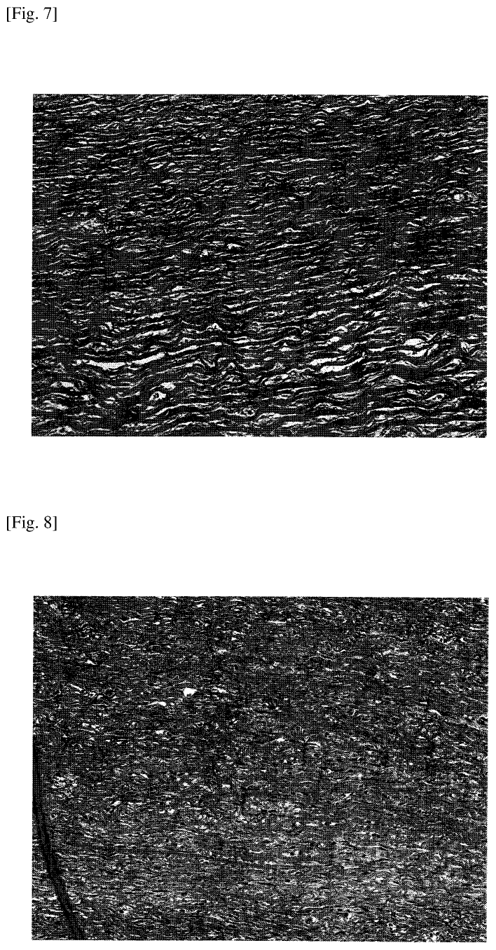

FIGS. 5-8 are images of hematoxylin-eosin stained (HE stained) samples of the decellularized tissues obtained in Examples 1-3 and the porcine aortas 57 as an untreated biological tissue.

It can be appreciated from FIGS. 5-8 that the decellularized tissues of Examples 1-3 are substantially free of damage and have no nucleus, which is present in the untreated biological tissue.

Table 1 indicates the amount of DNA per dry weight and the DNA length in the decellularized tissues of Examples 1-4 and the untreated biological tissue.

TABLE 1

| | | | DNA | |
|---|---|---|---|---|
| | | | AMOUNT | |
| | | ENZYMATIC | PER DRY | DNA |
| | | TREATMENT | WEIGHT | LENGTH |
| | ENTRAINER | TIME | [ng/mg] | [bp] |
| EXAMPLE 1 | NO | 7 DAYS | 2 | <100 |
| EXAMPLE 2 | NO | 5 DAYS | 5 | — |
| EXAMPLE 3 | NO | 3 DAYS | 37 | — |
| EXAMPLE 4 | YES | 7 DAYS | 2 | — |
| UNTREATED | — | — | 2295 | 35000< |
| BIOLOGICAL | | | | |
| TISSUE | | | | |

Note that DNA was extracted from the decellularized tissue or the untreated biological tissue using PureLink Genomic DNA Kits (manufactured by Thermo Fisher Scientific Inc.). Also, the amount of DNA was determined through ultraviolet absorbance measurement using an ultra-trace spectrophotometer, Nano Drop 2000c (manufactured by Thermo Fisher Scientific Inc.). Further, the DNA length was determined through electrophoresis.

It can be appreciated from Table 1 that the decellularized tissues of Examples 1-4 satisfy the criterion that the DNA amount per dry weight be less than 50 ng/mg as described in Non-Patent Literature Document 1. Also, in the decellularized tissue of Example 1, the DNA length is less than 100 bp.

Although the present invention has been described above with reference to certain illustrative embodiments and examples, the present invention is not limited to these embodiments and examples, and numerous variations and modifications may be made without departing from the scope of the present invention.

The present application is based on and claims the benefit of the priority date of Japanese Patent Application No. 2017-095004 filed on May 11, 2017, with the Japanese Patent Office, the entire contents of which are hereby incorporated by reference.

### REFERENCE SIGNS LIST

1 storage tank
2 liquefied dimethyl ether
3 pump
4, 9, 13, 15, 18, 21, 22 valve
5, 10, 12, 14, 16, 19, 20, 23 conduit
6 extraction tank
7 biological tissue
8 filter
11 separation tank
17 condenser
100 decellularization pretreatment apparatus

1-10. (canceled)

11. A method for producing a decellularized tissue, the method comprising:
lysing a cell of a biological tissue using a liquid containing a liquefied gas; and
degrading a nucleic acid component contained in the lysed cell of the biological tissue using a nucleolytic enzyme.

12. The method for producing a decellularized tissue according to claim 11, wherein the liquid further contains a solvent.

13. The method for producing a decellularized tissue according to claim 12, wherein the solvent is as least one of water and ethanol.

14. The method for producing a decellularized tissue according to claim 11, wherein
the liquefied gas is at a temperature ranging from 1° C. to 40° C.

15. The method for producing a decellularized tissue according to claim 11, wherein
the liquefied gas is at a pressure ranging from 0.2 MPa to 5 MPa.

16. The method for producing a decellularized tissue according to claim 11, further comprising:
washing the biological tissue that has the nucleic acid component degraded.

17. The method for producing a decellularized tissue according to claim 16, wherein
the biological tissue that has the nucleic acid component degraded is washed with at least one of water, a physiological saline solution, an aqueous ethanol solution, and the liquid containing the liquefied gas.

18. The method for producing a decellularized tissue according to claim 17, wherein
the biological tissue that has the nucleic acid component degraded is washed with an ethanol aqueous solution or the liquid containing the liquefied gas and then washed with water or the physiological saline solution.

19. A decellularized tissue produced by the method according to claim 11, the decelluralized tissue containing DNA at an amount per dry weight of less than 50 ng/mg.

20. An apparatus for producing a decellularized tissue, the apparatus comprising:
a lysis unit for lysing a cell of a biological tissue using a liquid containing dimethyl ether; and
a degradation unit for degrading a nucleic acid component contained in the lysed cell of the biological tissue using a nucleolytic enzyme.
